(19) United States
(12) Patent Application Publication
(10) Pub. No.: US 2010/0215743 A1
(43) Pub. Date: Aug. 26, 2010

(54) Composition and drug delivery of bisphosphonates

(76) Inventor: Thomas W. Leonard, Wilmington, NC (US)

Correspondence Address: MYERS BIGEL SIBLEY & SAJOVEC, PO BOX 37428, RALEIGH, NC, 27627, US

(21) Appl. No.: 12/712,527

(22) Filed: Feb. 25, 2010

Related U.S. Application Data
(60) Provisional application No. 61/155,269, filed on Feb. 25, 2009.

(51) Int. Cl. A61K 31/675; A61P 19/02; A61P 19/08; A61P 35/00; A61P 35/04; A61K 9/22

(52) U.S. Cl. 424/468; 514/94

(57) ABSTRACT

The present invention provides methods of treating or preventing a medical condition that is responsive to a bisphosphonate compound in a subject. The methods comprise administering to the subject a pharmaceutical composition comprising a therapeutically effective amount of the bisphosphonate no less frequently than a bi-weekly dosage schedule. In some embodiment, the bisphosphonate compound is zoledronic acid.

### RELATED APPLICATIONS

This application claims the benefit under 35 U.S.C. §119(e) of U.S. Provisional Patent Application Ser. No. 61/155,269, filed Feb. 25, 2009, the disclosures of which is incorporated herein by reference in its entirety.

### FIELD OF THE INVENTION

The present invention generally relates to the compositions of bisphosphonates and the methods of treating medical conditions by using pharmaceutical composition comprising a bisphosphonate compound.

### BACKGROUND OF THE INVENTION

Bisphosphonates are an important class of drugs that has demonstrated promising effects in treating diseases associated with abnormally accelerated bone resorption such as osteoporosis, Paget's disease, tumor induced hypercalcaemia and more recently, bone metastases.

The doses required for treating tumor induced diseases are usually higher than those required for other treatments. For example, zoledronic acid, a bisphosphonate compound, may be used to treat osteoporosis, Paget's disease, hypercalcemia, bone metastases, or multiple myeloma. However, the dosage for treating oncology related diseases such as tumor induced hypocalcemia is about ten times higher than the dosage used for treating osteoporosis or related diseases. In addition, the absorption of bisphosphonates in the patient is very limited. Usually, less than 1% of the bisphosphonate active ingredient contents of a tablet may be absorbed. Furthermore, most bisphosphonates are well known to be toxic to the gastrointestinal (GI) tract.

Therefore, in order to reach the high dose of bisphosphonate required for oncology treatments, most treatments are carried out by intravenous infusion, which is inconvenient and expensive for patients. Intravenous bisphosphonate therapy (e.g. zoledronic acid) for osteoporosis is usually administered only once a quarter or a year due to the inconvenience and the cost associated with intravenous infusion therapy that must be used to achieve the required therapeutic effects. Oncology treatments using bisphosphonate, (e.g. zoledronic acid) are usually administered every 4 weeks, or in some very severe cases, once every 3 weeks. Similarly, the inconvenience and cost of therapy have driven these dosage schedules. Therefore, it is difficult to provide a sustained therapeutic effect by intravenous infusion therapy. In addition, patients may suffer infusion related side effects from the intravenous infusion. Some of the known oral administration methods may allow administration of a bisphosphonate compound with the high doses required for oncology treatment, however, damage to the GI tract is likely to occur due to the residue of unabsorbed drug from the high dose treatment. Furthermore, in addition to the potential damage to the GI tract, the high dosage of the bisphosphonate compound may also cause possible renal damage, fever, and a general malaise, particularly when the bisphosphonate is administered via intravenous infusion.

### SUMMARY OF THE INVENTION

Usually, the dosage for bisphosphonate therapy (e.g. zoledronic acid concentrate for intravenous infusion) for osteoporosis related conditions is about 10% of the dosage for oncology treatment. For the treatment of osteoporosis related conditions, the bisphosphonate may be administered 5 mg annually. For prevention of osteoporosis related condition, the bisphosphonate may be administered as 5 mg every other year. For the treatment of oncology related conditions, the bisphosphonate may be administered 4 mg every four weeks. The bisphosphonate has serious toxicity when administered as an intravenous infusion, including kidney toxicity, and acute phase syndrome, which includes fever and bone pain. This is particularly true for oncology treatment. As all bisphosphonates have appreciable GI toxicity associated with oral administration, zoledronic acid has never been given in a more frequently dosage scheme. In some severe oncology cases, the bisphosphonate is given as 4 mg every 3 weeks, which increases potential for toxicity.

One aspect of the invention provides methods of treatment or prevention to a subject having a medical condition that is responsive to a bisphosphonate compound. The methods comprise administering to the subject a pharmaceutical composition comprising a therapeutically effective amount of the bisphosphonate no less frequently than a bi-weekly dosage schedule, or in some embodiments, a weekly or daily dosage schedule. In some embodiments, the bisphosphonate compound is zoledronate. In one embodiment, the bisphosphonate is orally administered to the subject. In one embodiment, the methods described herein provide sustained therapeutic effects of the bisphosphonate. In another embodiment, the methods described herein provide reduced adverse effects resulting from administering a bisphosphonate compound to the subject.

In one embodiment, the medical conditions are selected from osteoporosis, rheumatoid arthritis, bone fracture, excessive bone resorption and a combination thereof. In another embodiment, the medical conditions are selected from systemic lupus erythematosus (SLE), cancer, tumor induced hypocalcemia, bone metastasis and a combination thereof. In one embodiment, the cancer is selected from the group consisting of prostate cancer, metastatic bone cancer, lung cancer, multiple myeloma, breast cancer and any solid tumor that induces metastatic disease.

In another embodiment, the pharmaceutical composition is in a solid oral dosage form. In some embodiments, the pharmaceutical composition further comprises an enhancer. In one embodiment, the enhancer is a medium chain fatty acid salt, an ester, an ether, or a derivative of a medium chain fatty acid and has a carbon chain length of from about 4 to about 20 carbon atoms. In one embodiment, the carbon chain length of the enhancer is from 6 to 20 or 8 to 14 carbon atoms. In one embodiment, the enhancer is selected from the group consisting of sodium caprylate, sodium caprate, sodium laurate and a combination thereof. In one embodiment, the enhancer is sodium caprate.

Objects of the present invention will be appreciated by those of skill in the art from a reading of the Figures and the detailed description of the embodiments which follow, such description being merely illustrative of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 graphically demonstrates the correlation of serum C-telopeptide (CTX) of cohorts A, B and C over time.

FIG. 2 graphically demonstrates the correlation of N-Telopeptide Cross-Links (NTx) in Urine of cohorts A, B and C over time.

FIG. 3 graphically demonstrates the comparison of the calcium level of cohorts A, B and C over time.

FIG. 4 graphically demonstrates the correlation of bone specific alkaline phosphatase of cohorts A, B and C over time.

FIGS. 5(a) and (b) shows the pain inventory for the three dosage schedules with average severity and worst severity.

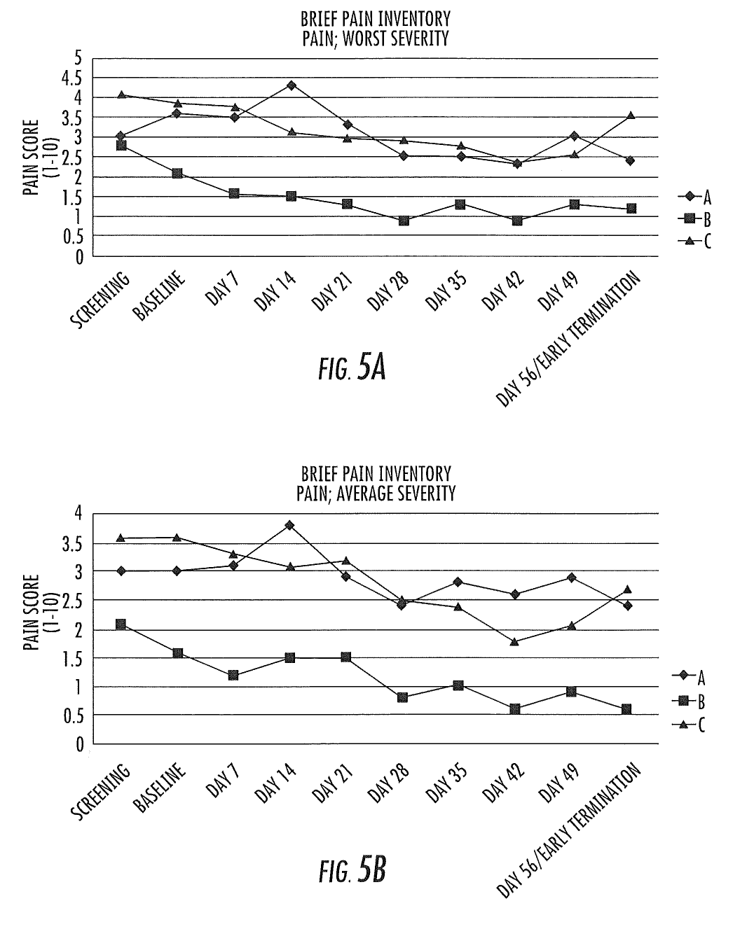

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of skill in the art to which this invention belongs.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

All patents, patent applications and publications referred to herein are incorporated by reference in their entirety. In case of a conflict in terminology, the present specification is controlling.

The investigators of the present invention have identified that alternate dosage schedules may be used to provide substantially improved therapeutic effects. These improvements may include reducing adverse effects resulting from administering a bisphosphonate compound and/or providing sustained therapeutic effects.

One aspect of the invention provides methods of treating or preventing a medical condition that is responsive to a bisphosphonate compound in a subject. The methods comprise administering to the subject a pharmaceutical composition comprising a therapeutically effective amount of the bisphosphonate no less frequently than a biweekly dosage schedule.

As used herein, “a medical condition that is responsive to a bisphosphonate compound” refers to medical conditions that may be treated or prevented by administering a bisphosphonate compound. Exemplary medical conditions include, but are not limited to, osteoporosis, rheumatoid arthritis, bone fracture, excessive bone resorption and a combination thereof. Further exemplary medical conditions include, but are not limited to, SLE, cancer (e.g., prostate cancer, metastatic bone cancer, lung cancer, multiple myeloma breast cancer and any solid tumor that induces metastatic disease), tumor induced hypocalcemia, bone metastasis and a combination thereof.

As used herein, “treat”, “treatment”, “treating” refer to reversing, alleviating, or inhibiting the progress of a medical condition, disorder or disease as described herein.

As used herein, “prevention”, “prevent”, and “preventing” refer to eliminating, reducing or delaying the incidence or onset of a medical condition, a disorder or disease as described herein, as compared to that which would occur in the absence of the measures taken.

In some embodiments, the bisphosphonate is administered to the subject via intravenous administration. In another embodiment, the bisphosphonate is orally administered to the subject.

In one embodiment, the treatment or prevention described herein may provide sustained therapeutic effects of the bisphosphonate. As used herein, “sustained therapeutic effect” refers to a relatively constant efficacy level of the bisphosphonate compound in the administered subject. In some embodiments, the sustained therapeutic effect is reflected by the relatively sustained level of the applicable biomarkers, for example, the fluctuations of the biomarkers is no more than about 5%, 10%, 20% or 30% of the mean level of the biomarkers during the treatment. As used herein, “during the treatment” is the period that the bisphosphonate is periodically administered to the subject. Any applicable biomarkers may be used in the present invention, e.g., those biomarkers associated with bone metabolism. Exemplary biomarkers include, but are not limited to, bone alkaline phosphatase, N-Telopeptide Cross-Links (NTX) in urine, serum C-telopeptide (CTX), or serum calcium level.

In one embodiment, after administering the pharmaceutical compositions described herein to a subject, the level of NTX in urine in the subject is decreased and maintained in a range of about 5 to about 60 BCE/mMol, about 1 to about 41 BCE/mMol, about 11 to about 31 BCE/mMol or, about 15 to about 35 BCE/mMol during the treatment. As used herein, “BCE/mmol” is bone collagen equivalent per mill mole. In another embodiment, the level of NTX in urine in the subject is decreased and maintained in a range of about 20 to about 30 BCE/mMol during the treatment. In some embodiments, the decrease fluctuations of NTX is no more than about 5%, 10%, 20% or 30% of the mean decreased level of the NTX.

In one embodiment, the level of CTX of the subject is decreased and maintained at a range of about 35 to about 600 pg/mL, about 100 to about 300 pg/mL, or about 5 to about 350 pg/mL during the treatment. As used herein, “pg/ml” is pictogram per milliliter. In another embodiment, the level of CTX of the subject is decreased and maintained at a range of about 150-about 260 pg/mL during the treatment. In some embodiments, the decrease fluctuations of CTX is no more than 5%, 10%, 20% or 30% of the mean decreased level of the CTX.

In another embodiment, the methods described herein may provide reduced adverse effects resulting from administering a bisphosphonate compound to the subject. As used herein, “reduced adverse effects” refers to a reduction in frequency and/or severity of adverse effects compared to a bisphosphonate compound administered via a method commonly used in the market (e.g., IV infusion) on a monthly or yearly dosage schedule. The adverse effect may be any toxic or side effects resulting from administering the bisphosphonate compound. In one embodiment, the adverse effect is selected from renal damage, general malaise, acute phase reaction, stomach pain, fatigue, nausea, or a combination thereof. In another embodiment, the acute phase reaction is selected from fever, muscle pain, bone pain, or a combination thereof.

In one embodiment, the bisphosphonate is administered to the subject on a weekly dosage schedule or a daily dosage schedule. In another embodiment, when the pharmaceutical composition is administered orally, the oral dose of the bisphosphonate compound is about 8 to 400 times or 8 to about 200 times more than the systemic dose of bisphosphonate compound administered through intravenous infusion. As used herein, “systemic dose” refers to the amount of a bisphosphonate compound delivered to the circulatory system of a subject via either intravenous infusion or oral administration. As used herein, “oral dose” refers to the amount of a bisphosphonate compound in an oral dosage form of the bisphosphonate compound, for example, the amount of the bisphosphonate compound in one or more tablets or capsules.

In some embodiments, the methods described herein are used to treat or prevent osteoporosis related conditions such as osteoporosis, rheumatoid arthritis, bone fracture, excessive bone resorption or a combination thereof. When the methods described herein are used to treat osteoporosis related medical conditions, the systemic dose of the pharmaceutical composition is in a range of about 0.000018 mmol (e.g., 0.005 mg zoledronic acid) to about 0.00015 mmol (e.g., 0.04 mg zoledronic acid) of the bisphosphonate compound per day. In another embodiment, the systemic dose of the pharmaceutical composition is in a range of about 0.00013 mmol (e.g., 0.035 mg zoledronic acid) to about 0.001 mmol (e.g., 0.28 mg zoledronic acid) of the bisphosphonate compound per week. In one embodiment, when the bisphosphonate (e.g., zoledronic acid) is administered in a dosage form of a tablet on a weekly dosage schedule and the bioavailability of the tablet is about 5%, the oral dosage of the bisphosphonate compound is in a range of about 0.0026 mmol (e.g., 0.7 mg zoledronic acid) to about 0.02 (e.g., 5.6 mg zoledronic acid). In one embodiment, when the bisphosphonate (e.g., zoledronic acid) is administered in a dosage form of a tablet on a biweekly dosage schedule and the bioavailability of the tablet is about 5%, the oral dose of the bisphosphonate compound is in a range of about 0.005 mmol (e.g., 1.4 mg zoledronic acid) to about 0.04 (e.g., 11.2 mg zoledronic acid). In another embodiment, when the bisphosphonate (e.g., zoledronic acid) is administered in a dosage form of a tablet on a daily dosage schedule and the bioavailability of the tablet is about 5%, the oral dose of the bisphosphonate compound is in a range of about 0.00037 mmol (e.g., 0.1 mg zoledronic acid) to about 0.0028 (e.g., 0.8 mg zoledronic acid). The ranges provided herein are intended to provide exemplary ranges of the oral dose for bisphosphonate in a tablet dosage form. However, the oral dose may vary when the bioavailability of the tablet changes.

In another embodiment, the methods described herein are used to treat oncology related conditions, for example, but are not limited to, systemic lupus erythematosus (SLE), cancer, tumor induced hypocalcemia, bone metastasis or a combination thereof. In some embodiments, the cancer is any solid tumor that may induce bone metastatic diseases. In one embodiment, the cancer is selected from prostate cancer, metastatic bone cancer, lung cancer, multiple myeloma, breast cancer and any solid tumor that induces metastatic disease. When the methods described herein are used to treat oncology related conditions, the systemic dose of the pharmaceutical composition is in a range of about 0.00018 mmol (e.g., 0.05 mg zoledronic acid) to about 0.0015 mmol (e.g., 0.4 mg zoledronic acid) of the bisphosphonate compound per day. In another embodiment, the systemic dose of the pharmaceutical composition is in a range of about 0.0013 mmol (e.g., 0.35 mg zoledronic acid) to about 0.01 mmol (e.g., 2.8 mg zoledronic acid) of the bisphosphonate compound per week. In one embodiment, when the bisphosphonate (e.g., zoledronic acid) is administered in a dosage form of a tablet on a weekly dosage schedule and the bioavailability of the tablet is about 5%, the oral dosage of the bisphosphonate compound is in a range of about 0.026 mmol (e.g., 7 mg zoledronic acid) to about 0.2 (e.g., 56 mg zoledronic acid). In one embodiment, when the bisphosphonate (e.g., zoledronic acid) is administered in a dosage form of a tablet on a biweekly dosage schedule and the bioavailability of the tablet is about 5%, the oral dose of the bisphosphonate compound is in a range of about 0.05 mmol (e.g., 14 mg zoledronic acid) to about 0.4 (e.g., 112 mg zoledronic acid). In another embodiment, when the bisphosphonate (e.g., zoledronic acid) is administered in a dosage form of a tablet on a daily dosage schedule and the bioavailability of the tablet is about 5%, the oral dose of the bisphosphonate compound is in a range of about 0.0037 mmol (e.g., 1 mg zoledronic acid) to about 0.028 (e.g., 8 mg zoledronic acid). The ranges provided herein are intended to provide exemplary ranges of the oral dosage for bisphosphonate in a tablet dosage form. However, the oral dosage may vary when the bioavailability of the tablet changes.

According to some embodiments of the present invention, when the pharmaceutical composition of the bisphosphonate compound is administered at the dosage schedule described herein, the sustained therapeutic effect and reduced adverse effects may be provided with or without the enhancers described herein and the pharmaceutical composition may be administered via any applicable administration methods.

It is understood that a specific dose level for any particular subject may depend upon a variety of factors including the activity of the specific bisphosphonate compound employed, the age, body weight, general health, sex, diet, time of administration, rate of excretion, drug combination, and the severity of the particular disease being treated and form of administration. It is further understood that the ordinarily skilled physician or veterinarian will readily determine and prescribe the effective amount of the bisphosphonate compound for prophylactic or therapeutic treatment of the condition for which treatment is administered.

The terms “bisphosphonate”, as used herein, include acids, salts, esters, hydrates, polymorphs, hemihydrates, solvates, and derivatives of the bisphosphonate compound. Non-limiting examples of bisphosphonates useful herein include the following:

(a) Alendronate, also known as Alendronic acid, 4-amino-1-hydroxybutylidene-,1-bisphosphonic acid, alendronate sodium, alendronate monosodium trihydrate or 4-amino-1-hydroxybutylidene-1,1-bisphosphonic acid monosodium trihydrate;

(b) [(cycloheptylamino)-methylene]-bis-phosphonate (incadronate);

(c) (dichloromethylene)-bis-phosphonic acid (clodronic acid) and the disodium salt (clodronate);

(d) [1-hydroxy-3-(1-pyrrolidinyl)-propylidene]-bis-phosphonate (EB-1053);

(e) (1-hydroxyethylidene)-bis-phosphonate (etidronate);

(f) [1-hydroxy-3-(methylpentylamino)propylidene]-bis-phosphonate (ibandronate);

(g) (6-amino-1-hydroxyhexylidene)-bis-phosphonate (neridronate);

(h) [3-(dimethylamino)-1-hydroxypropylidene]-bis-phosphonate (olpadronate);

(i) (3-amino-1-hydroxypropylidene)-bis-phosphonate (pamidronate);

(j) [2-(2-pyridinyl)ethylidene]-bis-phosphonate (piridronate);

(k) [1-hydroxy-2-(3-pyridinyl)-ethylidene]-bis-phosphonate (risedronate);

(l) {[(4-chlorophenyl)thio]methylene}-bis-phosphonate (tiludronate),

(m) Zoledronate also known as zoledronic acid, 1-hydroxy-2-(1H-imidazol-1-yl)ethylidene]-bis-phosphonate (zoledronate); and

(n) [1-hydroxy-2-imidazopyridin-(1,2-a)-3-ylethylidene]-bis-phospho-nate (minodronate).

In one embodiment of the invention, the bisphosphonate is selected from risedronate, alendronate, pamidronate, tiludronate, cimadronate, ibandronate, clodronate, or zoledronate. In one embodiment, the bisphosphonate is zoledronic acid.

As used throughout this specification and claims, the term “zoledronate or zoledronic acid” includes the related bisphosphonic acid forms, pharmaceutically acceptable salt forms, and equilibrium mixtures of these. The term “zoledronate” includes crystalline, hydrated crystalline, and amorphous forms of zoledronate and pharmaceutically acceptable salts.

The term “bisphosphonates” include all forms thereof including stereoisomers, enantiomers, diastereomers, racemic mixtures and derivatives thereof, for example, salts, acids, esters and the like. The bisphosphonate may be provided in any suitable phase state including as a solid, liquid, solution, suspension and the like. When provided in solid particulate form, the particles may be of any suitable size or morphology and may assume one or more crystalline, semi-crystalline and/or amorphous forms.

Non-limiting examples of bisphosphonate salts useful herein include those selected from the group alkali metal (e.g. sodium, potassium etc), alkaline metal, ammonium, and mono-, di-, tri-, or tetra C₁-C₃₀ alkyl-substituted ammonium.

The bisphosphonates that may be used in the present invention are further discussed in the U.S. Application Publication Nos. 2003/0139378 and 2004/0157799, which are incorporated by reference in their entireties.

The amount of bisphosphonate active ingredient contained in the oral dosage forms of the present invention will depend on the particular bisphosphonate selected and the dosage schedule upon which the bisphosphonate is dosed to the patient. The dosage schedules of daily, weekly, and biweekly are non-limiting examples of dosage regimens suitable for use with the oral dosage forms or intravenous infusion of the present invention. The term “biweekly” means that a dosage form is administered once every 14 days. The terms “weekly” means that a dosage form is administered once every 7 days. The term “daily” means that a dosage form is administered once every day.

As used herein, a “therapeutically effective amount” refers to an amount of a bisphosphonate that elicits a therapeutically useful response in treating an existing medical condition and/or preventing or delaying the onset of a medical condition from occurring in a subject. In some embodiments, the subject is a mammal. In some embodiments, the subject is a human.

In some embodiments, in the methods described herein, the bisphosphonate may be administered in an oral dosage form. In another embodiment, when the pharmaceutical composition is administered orally, the pharmaceutical composition may further comprise an enhancer. As used herein, the term “enhancer” refers to a compound (or a mixture of compounds) which is capable of enhancing the transport of a drug, such as a bisphosphonate compound, across the GI tract in a subject such as a human. In some embodiments, the enhancer is a medium chain fatty acid or a medium chain fatty acid derivative having a carbon chain length of from 4 to 20 carbon atoms, or 6 to 20 carbon atoms. In some embodiments, the enhancer is a medium chain fatty acid or a medium chain fatty acid derivative having a carbon chain length of from 6 to 20 carbon atoms with the provisos that (i) where the enhancer is an ester of a medium chain fatty acid, said chain length of from 6 to 20 carbon atoms relates to the chain length of the carboxylate moiety, and (ii) where the enhancer is an ether of a medium chain fatty acid, at least one alkoxy group has a carbon chain length of from 6 to 20 carbon atoms. In some embodiments, the enhancer is solid at room temperature and has a carbon chain length of from 8 to 14 carbon atoms. In another embodiment, the enhancer is a sodium salt of a medium chain fatty acid. In a further embodiment, the enhancer is sodium caprylate, sodium caprate, sodium laurate or a combination thereof. In some embodiments, the enhancer is sodium caprate. In another embodiment, the drug (bisphosphonate) and enhancer can be present in a ratio of from 1:100000 to 10:1 (drug (bisphosphonate):enhancer) or from 1:1000 to 10:1. The enhancers are further described in U.S. Pat. Nos., 7,658,938 and 7,670,626, and U.S. Patent Application Publication Nos. 2003/0091623 and 2007/0238707, which are incorporated by reference in their entirety.

As used herein, the term “medium chain fatty acid derivative” includes fatty acid salts, esters, ethers, acid halides, amides, anhydrides, carboxylate esters, nitrites, as well as glycerides such as mono-, di- or tri-glycerides. The carbon chain may be characterized by various degrees of saturation.

In one embodiment, the carbon chain may be fully saturated or partially unsaturated (i.e. containing one or more carbon-carbon multiple bonds). The term “medium chain fatty acid derivative” is referred to encompass also medium chain fatty acids wherein the end of the carbon chain opposite the acid group (or derivative) is also functionalized with one of the above mentioned moieties (i.e., an ester, ether, acid halide, amide, anhydride, carboxylate esters, nitrile, or glyceride moiety). Such difunctional fatty acid derivatives thus include for example diacids and diesters (the functional moieties being of the same kind) and also difunctional compounds comprising different functional moieties, such as amino acids and amino acid derivatives, for example a medium chain fatty acid or an ester or a salt thereof comprising an amide moiety at the opposite end of the fatty acid carbon chain to the acid or ester or salt thereof.

As used herein, a “therapeutically effective amount of an enhancer” refers to an amount of enhancer that enhances intestinal delivery of the drug such as a bisphosphonate compound to the underlying circulation and allows for the uptake of a therapeutically effective amount of the drug such as a bisphosphonate compound via oral administration. It has been shown that the effectiveness of an enhancer in enhancing the gastrointestinal delivery of poorly permeable drugs is dependent on the site of administration, the site of optimum delivery being dependent on the drug and enhancer.

The combination of bisphosphonates and enhancers is further described in U.S. Patent Application Publication No. 2007/0238707, which is incorporated by reference in its entirety.

In one embodiment, the pharmaceutical composition is in an oral dosage form, e.g., solid oral dosage form. The oral dosage form of bisphosphonates described in the present invention may deliver an effective amount of bisphosphonates to a patient quickly and without any of the deleterious side effects associated with intravenous infusion.

In one embodiment, the oral dosage form may be a tablet, a multiparticulate, or a capsule. In some embodiments, the oral dosage form is a delayed release dosage form which minimizes the release of drug and enhancer in the stomach, and hence the dilution of the local enhancer concentration therein, and releases the drug and enhancer in the intestine. In some embodiments, the oral dosage form is a delayed release rapid onset dosage form. Such a dosage form minimizes the release of drug and enhancer in the stomach, and hence the dilution of the local enhancer concentration therein, but releases the drug and enhancer rapidly once the appropriate site in the intestine has been reached, maximizing the delivery of the poorly permeable drug by maximizing the local concentration of drug and enhancer at the site of absorption.

As used herein, the term “tablet” includes, but is not limited to, immediate release (IR) tablets, sustained release (SR) tablets, matrix tablets, multilayer tablets, multilayer matrix tablets, extended release tablets, delayed release tablets and pulsed release tablets any or all of which may optionally be coated with one or more coating materials, including polymer coating materials, such as enteric coatings, rate-controlling coatings, semi-permeable coatings and the like. The term “tablet” also includes osmotic delivery systems in which a drug compound such as a bisphosphonate is combined with an osmagent (and optionally other excipients) and coated with a semi-permeable membrane, the semi-permeable membrane defining an orifice through which the drug compound may be released. Tablet solid oral dosage forms of the pharmaceutical composition used in the present invention include, but are not limited to, those selected from the group consisting of IR tablets, SR tablets, coated IR tablets, matrix tablets, coated matrix tablets, multilayer tablets, coated multilayer tablets, multilayer matrix tablets and coated multilayer matrix tablets. In some embodiments, the tablet dosage form is an enteric coated tablet dosage form. In another embodiment, the tablet dosage form is an enteric coated rapid onset tablet dosage form.

As used herein, the term “capsule” includes instant release capsules, sustained release capsules, coated instant release capsules, coated sustained release capsules, delayed release capsules and coated delayed release capsules. In one embodiment, the capsule dosage form is an enteric coated capsule dosage form. In another embodiment, the capsule dosage form is an enteric coated rapid onset capsule dosage form.

The term “multiparticulate” as used herein means a plurality of discrete particles, pellets, mini-tablets and mixtures or combinations thereof. If the oral form is a multiparticulate capsule, hard or soft capsule, e.g., gelatin capsules, can suitably be used to contain the multiparticulate. In one embodiment, a sachet can suitably be used to contain the multiparticulate. The multiparticulate may be coated with a layer containing rate controlling polymer material. The multiparticulate oral dosage form may comprise a blend of two or more populations of particles, pellets, or mini-tablets having different in vitro and/or in vivo release characteristics. For example, a multiparticulate oral dosage form may comprise a blend of an instant release component and a delayed release component contained in a suitable capsule. In one embodiment, the multiparticulate dosage form comprises a capsule containing delayed release rapid onset minitablets. In another embodiment, the multiparticulate dosage form comprises a delayed release capsule comprising instant release minitablets. In a further embodiment, the multiparticulate dosage form comprises a capsule comprising delayed release granules. In yet another embodiment, the multiparticulate dosage form comprises a delayed release capsule comprising instant release granules.

In another embodiment, the multiparticulate together with one or more auxiliary excipient materials may be compressed into tablet form such as a single layer or multilayer tablet. In some embodiments, a multilayer tablet may comprise two layers containing the same or different levels of the same active ingredient having the same or different release characteristics. In another embodiment, a multilayer tablet may contain a different active ingredient in each layer. The tablet, either single layered or multilayered, can optionally be coated with a controlled release polymer so as to provide additional controlled release properties.

In one embodiment, a multilayer tablet of the pharmaceutical composition used the present invention described herein is provided. In some embodiments, such a multilayer tablet may comprise a first layer containing a bisphosphonate and an enhancer in an instant release form and a second layer containing a bisphosphonate and an enhancer in a modified release form. As used herein, the term “modified release” includes sustained, delayed, or otherwise controlled release of a bisphosphonate upon administration to a patient. In an alternative embodiment, a multilayer tablet may comprise a first layer containing a bisphosphonate and a second layer containing an enhancer. Each layer may independently comprise further excipients chosen to modify the release of the bisphosphonate and/or the enhancer. Thus the bisphosphonate and the enhancer may be released from the respective first and second layers at rates which are the same or different. Alternatively, each layer of the multilayer tablet may comprise both a bisphosphonate and enhancer in the same or different amounts.

In yet another embodiment, a multiparticulate of the pharmaceutical composition used in the present invention is provided. The multiparticulate may comprise particles, pellets mini-tablets or combinations thereof, and the bisphosphonate and the enhancer may be contained in the same or different populations of particles, pellets or minitablets making up the multiparticulate. In another embodiment, multiparticulate, sachets and capsules such as hard or soft gelatin capsules may suitably be used to contain the multiparticulate. A multiparticulate dosage form may comprise a blend of two or more populations of particles, pellets or minitablets having different in vitro and/or in vivo release characteristics. For example, a multiparticulate dosage form may comprise a blend of an immediate release component and a delayed release component contained in a suitable capsule.

In the case of any of the embodiments described herein, a controlled release coating may be applied to the final dosage form (capsule, tablet, multilayer tablet etc.). In one embodiment, the controlled release coating may comprise a rate controlling polymer material as defined below. The dissolution characteristics of such a coating material may be pH dependent or independent of pH.

As used herein, the term “rate controlling polymer material” includes hydrophilic polymers, hydrophobic polymers and mixtures of hydrophilic and/or hydrophobic polymers that are capable of controlling or retarding the release of the drug compound from a solid oral dosage form of the present invention. Suitable rate controlling polymer materials include those selected from the group consisting of hydroxyalkyl cellulose such as hydroxypropyl cellulose and hydroxypropyl methyl cellulose; poly(ethylene) oxide; alkyl cellulose such as ethyl cellulose and methyl cellulose; carboxymethyl cellulose, hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate) and poly(vinyl acetate). Other suitable hydrophobic polymers include polymers and/or copolymers derived from acrylic or methacrylic acid and their respective esters, zein, waxes, shellac and hydrogenated vegetable oils.

Particularly useful in the practice of the present invention are poly acrylic acid, poly acrylate, poly methacrylic acid and poly methacrylate polymers such as those sold under the Eudragit® trade name (Rohm GmbH, Darmstadt, Germany) specifically Eudragit® L, Eudragit® S, Eudragit® RL, Eudragit® RS coating materials and mixtures thereof. Some of these polymers can be used as delayed release polymers to control the site where the drug is released. They include polymethacrylate polymers such as those sold under the Eudragit™ trade name (Rohm GmbH, Darmstadt, Germany) specifically Eudragit® L, Eudragit® S, Eudragit® RL, Eudragit® RS coating materials and mixtures thereof.

The various embodiments of the oral dosage forms of the pharmaceutical composition used in the present invention may further comprise auxiliary excipient materials such as, for example, diluents, lubricants, disintegrants, plasticizers, anti-tack agents, opacifying agents, pigments, flavorings and the like. As will be appreciated by those skilled in the art, the exact choice of excipients and their relative amounts will depend to some extent on the final dosage form.

Suitable diluents include for example pharmaceutically acceptable inert fillers such as microcrystalline cellulose, lactose, dibasic calcium phosphate, saccharides, and/or mixtures of any of the foregoing. Examples of diluents include microcrystalline cellulose such as that sold under the Avicel trademark (FMC Corp., Philadelphia, Pa.) for example Avicel™ pH101, Avicel™ pH102 and Avicel™ pH112; lactose such as lactose monohydrate, lactose anhydrous and Pharmatose DCL21; dibasic calcium phosphate such as Emcompress® (JRS Pharma, Patterson, N.Y.); mannitol; starch; sorbitol; sucrose; and glucose.

Suitable lubricants, including agents that act on the flowability of the powder to be compressed are, for example, colloidal silicon dioxide such as Aerosil™ 200; talc; stearic acid, magnesium stearate, and calcium stearate.

Suitable disintegrants include for example lightly cross-linked polyvinyl pyrrolidone, corn starch, potato starch, maize starch and modified starches, croscarmellose sodium, cross-povidone, sodium starch glycolate and combinations and mixtures thereof.

The weight and size of oral dosage form may be adjusted to meet required systemic doses based on the percent of bioavailability of the bisphosphonate compound in the oral dosage form. Techniques for making these dose adjustments are known to one skilled in the art.

Another aspect of the present invention provides pharmaceutical formulations that comprise zoledronic acid, sodium decanoate, sorbitol, colloidal silicon dioxide, stearic acid, hydroxypropyl methylcellulose (e.g., opadry 1 yellow), enteric coating (e.g., Acryl-EZE II) and Talc. In one embodiment, the formulation is in a tablet dosage form.

The present invention will now be described in more detail with reference to the following examples. However, these examples are given for the purpose of illustration and are not to be construed as limiting the scope of the invention.

### EXAMPLES

### Example 1

### The Preparation of the Oral Dosage Form of Zoledronic Acid (Orazol™) and the Test of the Tablet

Immediate release tablets containing zoledronic acid are made by preparing a granulation containing about 20 mg active ingredient (zoledronic acid), the enhancer (sodium caprate) and other excipients. The granulation is compressed into tablets. The tablets are placed into a coating pan, and a standard enteric coating is applied to the tablets. Table 1 provides the content, and dissolution data for the tablets of zoledronic acid, and demonstrates that the tablets are appropriate for use in clinical trials. The data indicate that the tablets contained 20 mg of active ingredient. No release of the active ingredient occurs when the tablets are placed in acid, indicating the integrity of the enteric coating. The tablets fully release the active ingredient rapidly when they are placed in pH 6.8 buffer solution. Table 2 provides the formulation of Orazol™. Table 3 shows the dissolution rate of zoledronic acid and the enhancer, sodium caprate (C10) as well as stability test data. As shown in Table 3, the zoledronic acid and sodium caprate dissolve at a similar rate.

TABLE 1
Test data for Orazol ™ tablets

| Test | Specification | Results | |
|---|---|---|---|
| Appearance | White to off-white elliptical diamond | Conforms | |
| | shaped tablets | | |
| Assay | 18 mg to 22 mg of Zoledronic Acid | 19.7 mg | |
| Content Uniformity | Conforms to USP | Conforms, Min = 97.4%, | |
| | | Max = 104.8%, Mean = 1-1.9%, | |
| | | % RSD = 2.4%, AV = 6.4 | |
| Related Substances | NMT 0.5% of any individual impurity | None detected | |
| Dissolution: Acid Stage | NMT 10% per individual unit | Conforms, none detected in | |
| | | any of 6 units after 2 hours. | |
| Dissolution: Buffer Stage | Report Results for % released after 30 | Unit #1 | 79.6% |
| | minutes | Unit #2 | 56.8% |
| | | Unit #3 | 73.4% |
| | | Unit #4 | 65.5% |
| | | Unit #5 | 67.2% |
| | | Unit #6 | 57.5% |

TABLE 2
Formulation of Orazol (the enteric coating tablet of zoledronic acid)

| Component | Mg/tablet |
|---|---|
| Zoledronic Acid monohydrate | 21.32 equivalent to 20 mg zoledronic acid |
| Sodium Decanoate | 550.00 |
| Sorbitol | 274.68 |
| Colloidal silicon dioxide | 4.50 |
| Crospovidone | 45.00 |
| Stearic Acid | 4.5 |
| Opadry 1 yellow | 54.00 |
| Acryl-EZE II | 81.09 |
| Talc | 1.29 |

TABLE 3
Dissolution and Stability Test data for Orazol ™ tablets

| | | | 1 month | | 1 month | | 3 month | | 3 month | |
|---|---|---|---|---|---|---|---|---|---|---|
| | | | 25° C./60% | | 40° C./75% | | 25° C./60% | | 40° C./75% | |
| | 0 month | | RH | | RH | | RH | | RH | |
| Physical | Conforms | | Conforms | | Conforms | | Conforms | | Conforms | |
| Inspection | | | | | | | | | | |
| API Assay | 98.5% | | 99.5% | | 99.6% | | 99.6% | | 100.8% | |
| Related | ND | | ND | | ND | | ND | | ND | |
| Substances | | | | | | | | | | |
| Moisture | 2.4% | | 1.5% | | 1.2% | | 1.5% | | 1.3% | |
| Dissolution | Acid | ND | Acid | ND | Acid | ND | Acid | ND | Acid | ND |
| (zoledronic | 5 min | ND | 5 min | ND | 5 min | ND | 5 min | ND | 5 min | ND |
| acid, %) | 10 min | 1.7 | 10 min | 0.7 | 10 min | 0.4 | 10 min | 2.1 | 10 min | 1.6 |
| | 20 min | 30.4 | 20 min | 31.0 | 20 min | 28.8 | 20 min | 36.9 | 20 min | 34.6 |
| | 30 min | 66.7 | 30 min | 73.8 | 30 min | 65.4 | 30 min | 73.3 | 30 min | 74.7 |
| | 45 min | 95.0 | 45 min | 94.1 | 45 min | 92.1 | 45 min | 95.1 | 45 min | 99.1 |
| Dissolution | Acid | ND | Acid | ND | Acid | 0.1 | Acid | ND | Acid | ND |
| (C10, %) | 5 min | ND | 5 min | ND | 5 min | 0.2 | 5 min | 0.3 | 5 min | ND |
| | 10 min | 0.6 | 10 min | 2.6 | 10 min | 2.4 | 10 min | 2.6 | 10 min | 1.6 |
| | 20 min | 29.4 | 20 min | 31.7 | 20 min | 32.3 | 20 min | 37.8 | 20 min | 35.8 |
| | 30 min | 64.4 | 30 min | 75.4 | 30 min | 70.0 | 30 min | 74.2 | 30 min | 74.9 |
| | 45 min | 92.7 | 45 min | 95.8 | 45 min | 96.5 | 45 min | 96.0 | 45 min | 98.3 |

| | 6 month | | 6 month | | 12 month | | 18 month | |
|---|---|---|---|---|---|---|---|---|
| | 25° C./60% | | 40° C./75% | | 25° C./60% | | 25° C./60% | |
| | RH | | RH | | RH | | RH | |
| Physical | *Does not | | Conforms | | Conforms | | **Conforms | |
| Inspection | conform | | | | | | | |
| API Assay | 102.8% | | 102.7% | | 97.6% | | 96.3% | |
| Related | ND | | ND | | ND | | ND | |
| Substances | | | | | | | | |
| Moisture | 1.2% | | 1.9% | | 2.5% | | 1.6% | |
| Dissolution | Acid | ND | Acid | ND | Acid | ND | Acid | ND |
| (zoledronic | 5 min | ND | 5 min | ND | 5 min | ND | 5 min | ND |
| acid, %) | 10 min | 1.0 | 10 min | 3.7 | 10 min | 1.8 | 10 min | 0.6 |
| | 20 min | 31.8 | 20 min | 41.0 | 20 min | 26.7 | 20 min | 25.4 |
| | 30 min | 71.7 | 30 min | 78.5 | 30 min | 64.9 | 30 min | 60.2 |
| | 45 min | 96.6 | 45 min | 95.1 | 45 min | 94.1 | 45 min | 83.3 |
| Dissolution | Acid | ND | Acid | ND | Acid | ND | Acid | ND |
| (C10, %) | 5 min | ND | 5 min | ND | 5 min | ND | 5 min | ND |
| | 10 min | 2.0 | 10 min | 4.6 | 10 min | ND | 10 min | 0.9 |
| | 20 min | 31.5 | 20 min | 42.6 | 20 min | 28.8 | 20 min | 27.0 |
| | 30 min | 70.9 | 30 min | 78.9 | 30 min | 66.8 | 30 min | 62.4 |
| | 45 min | 96.6 | 45 min | 96.6 | 45 min | 96.0 | 45 min | 86.6 |

ND none detected
*PI failure. One cracked tablet observed and one tablet with sub-coat visible observed.
**Two out of twenty tablets showed minor defects. Determined not to be stability related.

### Example 2

### Comparison of Efficacy of Zometa® and Orazol™

### (1) Biomarkers

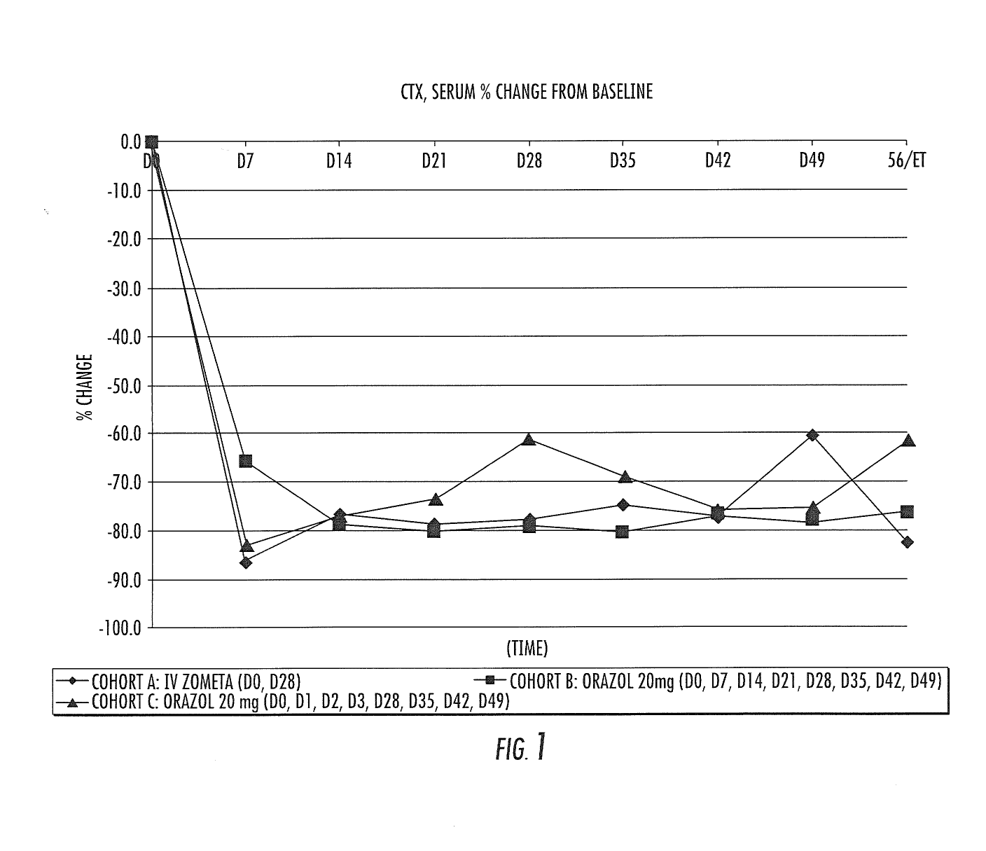

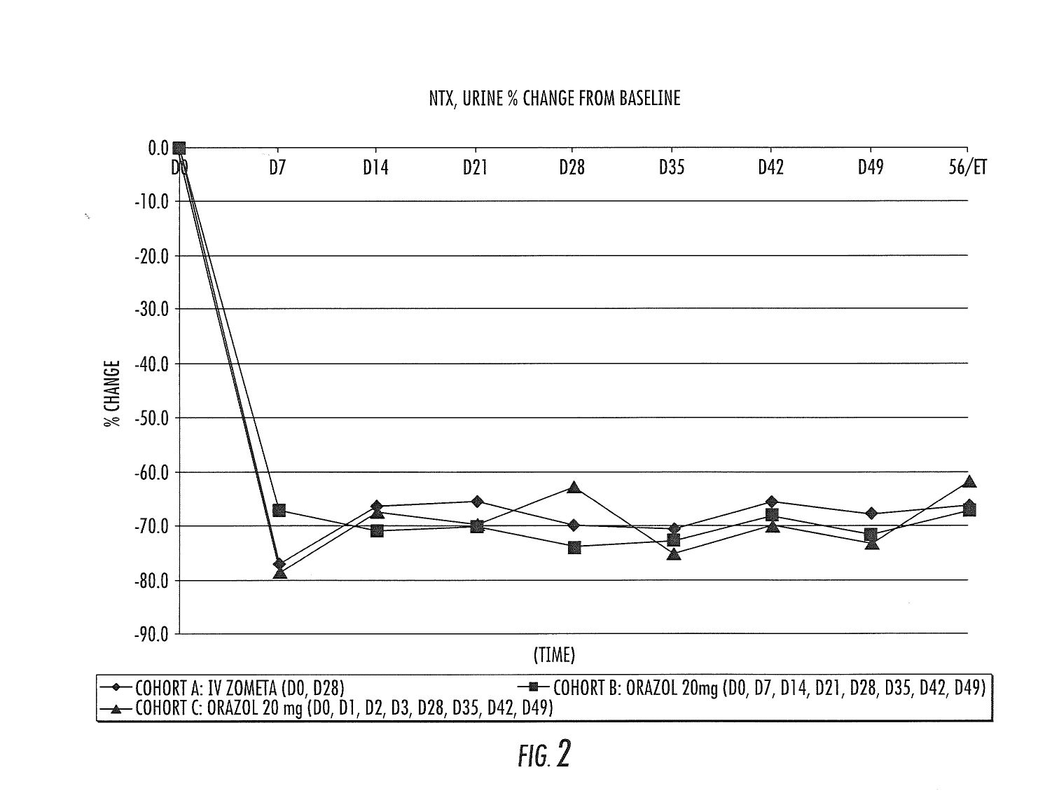

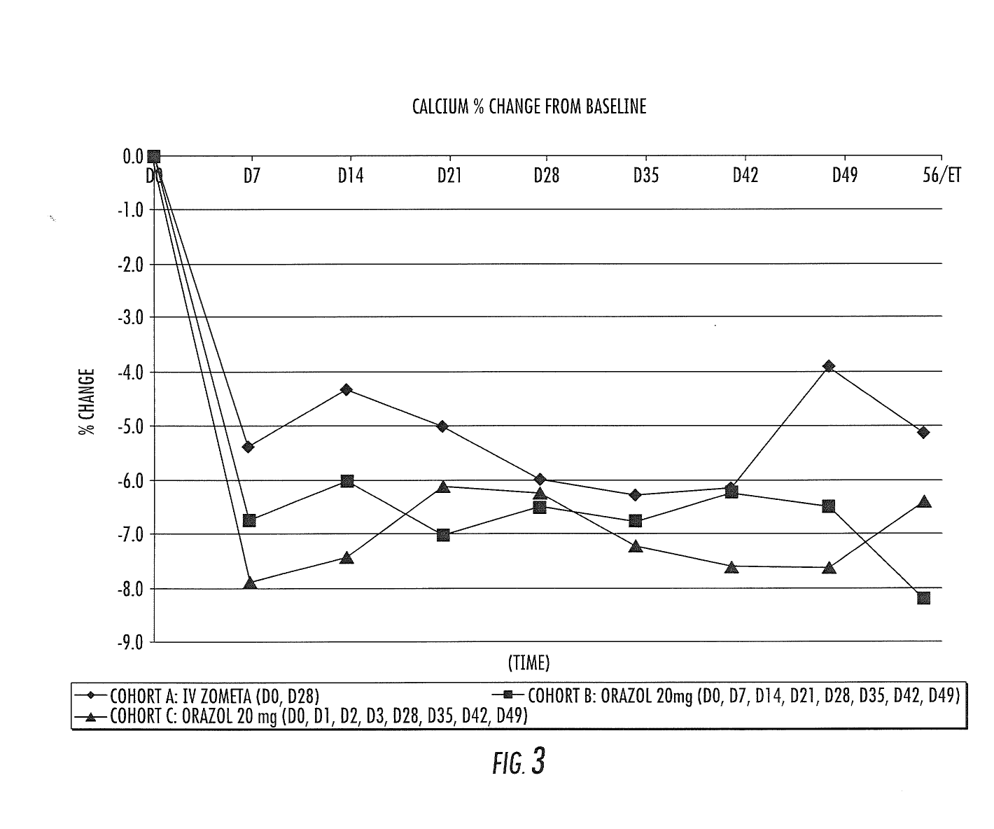

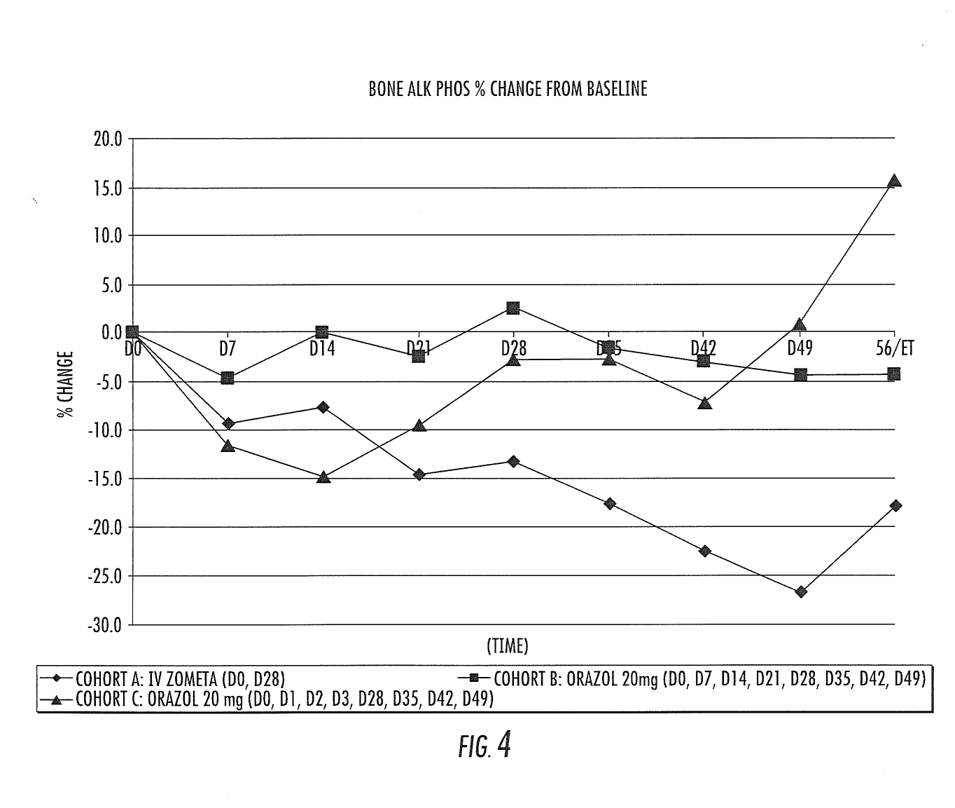

A clinical trial is carried out in hormone-refractory prostate cancer patients with evidence of bone metastasis using the tablets prepared in Example 1 and Zometa® concentrate for intravenous infusion, a commercially available form of zoledronic acid which can only be administered via intravenous infusion. It has been demonstrated that the 20 mg tablet delivers approximately 1 mg of zoledronic acid to the systemic circulation. Therefore, the administration of 4 tablets is equal to 4 mg administered by intravenous infusion, which is a normal dose used in oncology. Response to the treatment is monitored using biomarkers of bone metabolic activity for two dosage regimens of Orazol™ compared with Zometa® intravenous infusion. Thirty patients are enrolled in the study, and are divided into 3 groups. The group labeled as Cohort A receives a dose of 4 mg of Zometa® administered via intravenous infusion every 4 weeks, as indicated in the Zometa® product labeling, for a total of 8 weeks. Cohort B receives Orazol™ 20 mg tablets administered orally to patients once a week for a total of 8 weeks, Cohort C receives a loading dose of Orazol™ 20 mg tablets for the first 4 weeks of therapy. The loading dose is administered as 20 mg tablets every day for 4 days. Cohort C then receives weekly therapy of a 20 mg tablet each week for the second 4 weeks. Therefore, over the 8 weeks of the study all three groups receive equal doses of zoledronic acid systemically. To clarify, Cohort A corresponds to Zometa® 4 mg administered to the patients though intravenous infusion over 15 minutes on days 0 and 28. Cohort B corresponds to Orazol™ 20 mg administered orally to patients on days 0, 7, 14, 21, 28, 35, 42 and 49. Cohort C corresponds to Orazol™ 20 mg administered orally to patients on days 0, 1, 2, 3, 28, 35, 42 and 49. Four biomarkers such as bone alkaline phosphatase, CTX, calcium level and urine NTX, are tested at weekly intervals to determine the effects of the three treatments with different dosage. The biomarker data are shown below in Table 4 (a)-(d). FIGS. 1-4 graphically compared the biomarker data of Cohort A, B and C. Tables 5 (a)-(d) shows the changes for those four biomarkers from baseline.

FIGS. 1-4 demonstrate that bone metabolic markers respond to Orazol™ as rapidly and effectively as Zometa®. The responses to the biomarkers occur rapidly for both Cohort B and C. Furthermore, substantial mean decreases in urine NTX and serum CTX levels were observed in the three cohorts beginning at Day 7. Additionally, the examination of the data indicates that Cohort B provided a greater percent mean reduction of urine NTX and serum CTX at 5 out of 8 time points and overall was more consistent. Therefore, Cohort B trended towards better performance than Cohorts A and C in the reduction of these skeletal-related events (SRE) prognostic biomarkers, which indicates improved therapeutic effects.

TABLE 4(a)
serum C-telopeptide (CTX) data for Cohort A, B and C.
CTX, Serum

| Patient No. | | D0 | D7 | D14 | D21 | D28 | D35 | D42 | D49 | Cohort |
|---|---|---|---|---|---|---|---|---|---|---|
| 001 | 361 | 923 | 65 | 99 | 145 | 169 | 87 | 132 | 138 | A |
| 004 | 365 | 460 | <30 | 117 | <30 | <30 | 33 | <30 | 312 | A |
| 271 | | 1588 | 546 | 1240 | 931 | 1348 | 1592 | 1715 | 2383 | A |
| 369 | | 244 | 81 | 96 | 113 | | 113 | 63 | 105 | A |
| 392 | | 803 | 54 | 53 | 92 | 76 | 50 | 37 | 67 | A |
| 301 | | 339 | 34 | 51 | 38 | 72 | 71 | 40 | 48 | A |
| 332 | | 544 | 59 | 40 | | 71 | 61 | 52 | 53 | A |
| Cohort A | | 700 | 140 | 242 | 264 | 347 | 287 | 340 | 444 | |
| (Time) | | D0 | D7 | D14 | D21 | D28 | D35 | D42 | D49 | |
| SD | | 460 | 200 | 441 | 375 | 561 | 576 | 675 | 860 | |
| 002 | 362 | 521 | 35 | 64 | 55 | 85 | 90 | 87 | 58 | B |
| 005 | 364 | 587 | 155 | 230 | 146 | 117 | 117 | 112 | 129 | B |
| 368 | | 522 | | 100 | 42 | 118 | 97 | 106 | 107 | B |
| 391 | | 958 | 81 | 80 | 92 | 63 | 85 | 75 | 72 | B |
| 394 | | 1106 | 685 | 321 | 357 | 335 | 476 | 612 | 561 | B |
| 333 | | 479 | | 57 | 40 | 83 | 66 | 66 | 38 | B |
| 334 | | 507 | 148 | 96 | 185 | 108 | 105 | 106 | 118 | B |
| 213 | | 338 | 61 | 29 | 50 | 50 | 56 | 60 | 90 | B |
| 151 | | 1538 | 718 | 447 | 391 | 488 | 348 | 702 | 347 | B |
| Cohort B | | 728 | 269 | 158 | 151 | 161 | 160 | 214 | 169 | |
| SD | | 391 | 299 | 143 | 136 | 149 | 148 | 253 | 173 | |
| 003 | 363 | 533 | <30 | 119 | 130 | 411 | 335 | 393 | 222 | C |
| 367 | | 813 | 115 | 137 | 245 | 247 | | <30 | 120 | C |
| 393 | | 557 | 97 | 103 | 126 | 196 | 115 | 129 | 128 | C |
| 395 | | 1018 | 129 | | 196 | 202 | 152 | 120 | 143 | C |
| 302 | | 617 | 79 | 144 | 192 | 170 | 176 | 159 | 176 | C |
| 335 | | 1286 | 181 | 218 | 327 | 502 | 617 | 321 | 522 | C |
| 211 | | 375 | 86 | 98 | 80 | 107 | 127 | 109 | 82 | C |
| Cohort C | | 743 | 115 | 137 | 185 | 262 | 254 | 205 | 199 | |
| SD | | 318 | 37 | 44 | 83 | 142 | 195 | 121 | 149 | |

TABLE 4(b)
Data for N-Telopeptide Cross-Links (NTx) in Urine of cohort A, B and C
NTX, Urine

| Patient No. | | D0 | D7 | D14 | D21 | D28 | D35 | D42 | D49 | Cohort |
|---|---|---|---|---|---|---|---|---|---|---|
| 001 | 361 | 128 | 22 | 37 | 24 | 25 | 29 | 24 | 33 | A |
| 004 | 365 | 58 | 13 | 28 | 21 | 22 | 20 | 22 | 17 | A |
| 271 | | 937 | 230 | 365 | 463 | 306 | 375 | 414 | 496 | A |
| 369 | | 29 | 14 | 15 | 16 | 19 | 12 | 19 | 15 | A |
| 392 | | 73 | 11 | 19 | 15 | 15 | 14 | 23 | 19 | A |
| 301 | | 57 | 15 | 23 | 24 | 20 | 22 | 25 | 23 | A |
| 332 | | 41 | 7 | 7 | | 5 | 7 | 7 | 6 | A |
| Cohort A | | 189 | 45 | 71 | 94 | 59 | 68 | 76 | 87 | |
| (Time) | | D0 | D7 | D14 | D21 | D28 | D35 | D42 | D49 | |
| SD | | 331 | 82 | 130 | 181 | 109 | 135 | 149 | 181 | |
| 002 | 362 | 63 | 25 | 18 | 26 | 17 | 19 | 13 | 20 | B |
| 005 | 364 | 104 | 24 | 29 | 25 | 21 | 21 | 24 | 22 | B |
| 368 | | 63 | 25 | 27 | 22 | 16 | 19 | 25 | 24 | B |
| 391 | | 70 | 6 | 8 | 7 | | 9 | 9 | 7 | B |
| 394 | | 126 | 45 | 41 | 46 | 65 | 45 | 44 | 41 | B |
| 333 | | 57 | 17 | 16 | 22 | 13 | 26 | 27 | 19 | B |
| 334 | | 130 | 53 | 43 | 31 | 33 | 29 | 37 | 28 | B |
| 213 | | 38 | 10 | 10 | 10 | 9 | 11 | 16 | 10 | B |
| 151 | | 133 | 47 | 30 | 29 | 23 | 20 | 25 | 20 | B |
| Cohort B | | 87 | 28 | 25 | 24 | 25 | 22 | 24 | 21 | |
| SD | | 36 | 17 | 13 | 11 | 18 | 11 | 11 | 10 | |
| 003 | 363 | 185 | 29 | 77 | 43 | 94 | 15 | 61 | 51 | C |
| 367 | | 110 | 20 | 33 | 33 | 36 | | 40 | 28 | C |
| 393 | | 264 | 25 | 27 | 27 | 36 | 30 | 35 | 35 | C |
| 395 | | 125 | 19 | 18 | 26 | 30 | 23 | 15 | 15 | C |
| 302 | | 47 | 13 | 14 | 16 | 15 | 19 | 19 | 16 | C |
| 335 | | 175 | 34 | 30 | 45 | 64 | 59 | 34 | 57 | C |
| 211 | | 28 | 10 | 20 | 16 | 18 | 14 | 18 | 14 | C |
| Cohort C | | 133 | 21 | 31 | 29 | 42 | 27 | 32 | 31 | |
| SD | | 82 | 9 | 21 | 12 | 28 | 17 | 16 | 18 | |

TABLE 4(c)
Data of calcium level of cohort A, B and C
Calcium

| Patient No. | | D0 | D7 | D14 | D21 | D28 | D35 | D42 | D49 | Cohort |
|---|---|---|---|---|---|---|---|---|---|---|
| 001 | 361 | 2.28 | 2.06 | 2.16 | | 2.13 | 2.06 | 2.07 | 2.12 | A |
| 004 | 365 | 2.11 | 2.11 | 2.10 | 2.09 | 2.04 | 2.08 | 2.11 | 2.35 | A |
| 271 | | 2.40 | 2.21 | 2.24 | 2.20 | 2.14 | 2.09 | 2.14 | 2.14 | A |
| 369 | | 2.47 | 2.35 | 2.42 | 2.35 | 2.42 | 2.30 | 2.19 | 2.41 | A |
| 392 | | 2.29 | 2.14 | 2.18 | 2.21 | 2.15 | 2.19 | 2.13 | 2.18 | A |
| 301 | | 2.32 | 2.23 | 2.29 | 2.19 | 2.15 | 2.23 | 2.29 | 2.15 | A |
| 332 | | 2.39 | 2.24 | 2.18 | | 2.23 | 2.32 | 2.26 | 2.27 | A |
| Cohort A | | 2.32 | 2.19 | 2.22 | 2.21 | 2.18 | 2.18 | 2.17 | 2.23 | |
| (Time) | | D0 | D7 | D14 | D21 | D28 | D35 | D42 | D49 | |
| SD | | 0.12 | 0.10 | 0.11 | 0.09 | 0.12 | 0.11 | 0.08 | 0.11 | |
| 002 | 362 | 2.34 | 2.16 | 2.28 | | 2.23 | 2.15 | 2.20 | 2.12 | B |
| 005 | 364 | 2.31 | 2.09 | 2.16 | 2.19 | 2.13 | 2.14 | 2.16 | 2.14 | B |
| 368 | | 2.37 | 2.18 | 2.15 | 2.41 | 2.32 | 2.41 | 2.22 | 2.24 | B |
| 391 | | 2.35 | 2.23 | 2.25 | 2.21 | 2.16 | 2.19 | 2.24 | 2.17 | B |
| 394 | | 2.34 | 2.15 | 2.26 | 2.14 | 2.11 | 2.12 | 2.20 | 2.15 | B |
| 333 | | 2.50 | 2.23 | 2.30 | 2.35 | 2.39 | 2.28 | 2.29 | 2.30 | B |
| 334 | | 2.26 | 2.17 | 2.04 | 2.02 | 2.05 | 2.06 | 2.12 | 2.13 | B |
| 213 | | 2.43 | 2.23 | 2.33 | 2.20 | 2.28 | 2.23 | 2.30 | 2.30 | B |
| 151 | | 2.35 | 2.20 | 2.10 | 1.98 | 2.10 | 2.08 | 2.05 | 2.18 | B |
| Cohort B | | 2.36 | 2.18 | 2.21 | 2.19 | 2.20 | 2.18 | 2.20 | 2.19 | |
| SD | | 0.07 | 0.05 | 0.10 | 0.15 | 0.11 | 0.11 | 0.08 | 0.07 | |
| 003 | 363 | 2.55 | 2.29 | 2.34 | | 2.27 | 2.22 | 2.28 | 2.24 | C |
| 367 | | 2.42 | 2.26 | 2.22 | 2.25 | 2.32 | | | 2.23 | C |
| 393 | | 2.41 | 2.10 | 2.16 | 2.22 | 2.07 | 2.12 | 2.12 | 2.12 | C |
| 395 | | 2.44 | 2.16 | 2.11 | 2.21 | 2.15 | 2.14 | 2.18 | 2.14 | C |
| 302 | | 2.52 | 2.20 | 2.32 | 2.41 | 2.38 | 2.29 | 2.29 | 2.33 | C |
| 335 | | 2.24 | 2.11 | 2.04 | 2.08 | 2.14 | 2.22 | 2.10 | 2.06 | C |
| 211* | | 2.33 | 2.35 | 2.38 | 2.23 | 2.28 | 2.38 | 2.30 | 2.33 | C |
| Cohort C | | 2.42 | 2.21 | 2.22 | 2.23 | 2.23 | 2.23 | 2.21 | 2.21 | |
| SD | | 0.11 | 0.09 | 0.13 | 0.11 | 0.11 | 0.09 | 0.09 | 0.10 | |

TABLE 4(d)
Data of bone alkaline phosphatase (BAP) of cohort A, B and C
Bone Alk Phos

| Patient No. | | D0 | D7 | D14 | D21 | D28 | D35 | D42 | D49 | Cohort |
|---|---|---|---|---|---|---|---|---|---|---|
| 001 | 361 | 47.0 | 60.5 | 51.0 | 46.3 | 47.3 | 56.0 | 50.6 | 57.0 | A |
| 004 | 365 | 15.5 | 13.8 | 16.1 | 16.2 | 17.9 | 16.1 | 14.5 | 15.6 | A |
| 271 | | 299.7 | 208.4 | 237.4 | 173.8 | 143.7 | 175.4 | 209.5 | 207.3 | A |
| 369 | | 6.7 | 6.7 | 6.5 | 6.0 | 5.6 | 4.3 | 3.9 | 4.1 | A |
| 392 | | 13.8 | 13.1 | 13.7 | 13.2 | 13.9 | 12.0 | 8.9 | | A |
| 301 | | 17.7 | 12.6 | 12.8 | 11.6 | 12.2 | 10.6 | 11.1 | 9.8 | A |
| 332 | | 18.1 | 17.1 | 15.4 | | 14.9 | 15.1 | 17.6 | 20.3 | A |
| Cohort A | | 59.8 | 47.5 | 50.4 | 44.5 | 36.5 | 41.4 | 45.2 | 52.4 | |
| (Time) | | D0 | D7 | D14 | D21 | D28 | D35 | D42 | D49 | |
| SD | | 107 | 73 | 84 | 65 | 49 | 61 | 74 | 78 | |
| 002 | 362 | 62.7 | 57.1 | 75.6 | 77.3 | 100.3 | 114.8 | 109.3 | 122.6 | B |
| 005 | 364 | 19.4 | 16.0 | 17.7 | 17.5 | 19.6 | 16.4 | 15.3 | 12.9 | B |
| 368 | | 48.4 | 49.0 | 50.8 | 48.6 | 52.6 | 48.7 | 76.5 | 47.7 | B |
| 391 | | | 10.6 | 11.9 | 12.0 | 12.5 | 11.3 | 9.9 | 8.2 | B |
| 394 | | 38.0 | 34.5 | 36.8 | 46.7 | 42.2 | 48.4 | 44.2 | 55.6 | B |
| 333 | | 19.9 | 19.1 | 18.9 | 23.3 | 21.9 | 19.4 | 18.1 | 14.8 | B |
| 334 | | 37.7 | 34.3 | 37.8 | 30.5 | 28.5 | 21.8 | 20.7 | 22.2 | B |
| 213 | | 45.2 | 39.9 | 39.0 | 28.9 | 28.2 | 26.7 | 24.3 | 31.5 | B |
| 151 | | 40.8 | 45.7 | 51.7 | 45.8 | 50.0 | 47.0 | 49.3 | 50.9 | B |
| Cohort B | | 39.0 | 34.0 | 37.8 | 36.7 | 39.5 | 39.4 | 40.8 | 40.7 | |
| SD | | 14.4 | 15.9 | 20.1 | 20.1 | 26.6 | 31.8 | 33.3 | 35.4 | |
| 003 | 363 | 78.4 | 88.5 | 72.5 | 76.8 | 106.8 | 135.8 | 125.0 | 133.9 | C |
| 367 | | 27.7 | 28.9 | 19.2 | 22.2 | 21.3 | | 12.3 | 14.8 | C |
| 393 | | 35.2 | 25.9 | 22.4 | 28.3 | 35.3 | 35.2 | 45.3 | 54.6 | C |
| 395 | | 54.5 | 40.7 | 48.8 | 50.5 | 46.5 | 42.5 | 41.2 | 49.5 | C |
| 302 | | 17.3 | 12.2 | 14.4 | 14.8 | 18.4 | 17.7 | 18.7 | 22.4 | C |
| 335 | | 102.1 | 76.6 | 63.2 | 60.4 | 73.2 | 78.4 | 78.6 | 86.9 | C |
| 211 | | 10.9 | 11.6 | 11.8 | 11.0 | 11.3 | 11.9 | 10.6 | 9.9 | C |
| Cohort C | | 46.6 | 40.6 | 36.0 | 37.7 | 44.7 | 53.6 | 47.4 | 53.1 | |
| SD | | 33.6 | 30.5 | 25.0 | 25.1 | 34.4 | 46.6 | 41.7 | 44.7 | |

TABLE 5(a)
Changes from baseline in Serum CTX:

| Cohort A | | | Cohort B | | | Cohort C | | |
|---|---|---|---|---|---|---|---|---|
| (N = 8) | | | (N = 11) | | | (N = 10) | | |
| | | % | | | % | | | % |
| Actual | Change | Change | Actual | Change | Change | Actual | Change | Change |

Baseline

| | | | |
|---|---|---|---|
| N | 8 | 11 | 10 |
| Mean | 702.9 | 707.5 | 700.3 |
| SD | 425.86 | 379.56 | 270.91 |
| Median | 633.0 | 522.0 | 587.5 |
| Min, Max | 244, 1588 | 298, 1538 | 375, 1286 |

Day 7

| | | | | | | | | | |
|---|---|---|---|---|---|---|---|---|---|
| N | 8 | 8 | 8 | 9 | 9 | 9 | 10 | 10 | 10 |
| Mean | 108.6 | −594.3 | −86.55 | 256.2 | −497.3 | −65.74 | 114.2 | −586.1 | −83.00 |
| SD | 178.29 | 295.33 | 12.906 | 262.73 | 285.07 | 29.432 | 68.73 | 250.74 | 11.349 |
| Median | 56.5 | −596.0 | −91.46 | 148.0 | −432.0 | −73.59 | 106.0 | −524.5 | −85.89 |
| Min, Max | 15.0, 546.0 | −1042, −163 | −97.9, −65.6 | 35.0, 718.0 | −877, −11.0 | −93.3, −3.7 | 15.0, 243.0 | −1105, −289 | −97.2, −56.5 |

Day 56/Early Termination

| | | | | | | | | | |
|---|---|---|---|---|---|---|---|---|---|
| N | 6 | 6 | 6 | 11 | 11 | 11 | 10 | 10 | 10 |
| Mean | 87.8 | −508.0 | −82.50 | 176.8 | −530.7 | −76.08 | 272.3 | −428.0 | −61.40 |
| SD | 57.38 | 252.31 | 13.897 | 189.91 | 281.68 | 14.676 | 213.57 | 239.96 | 31.129 |
| Median | 74.0 | −559.0 | −86.38 | 107.0 | −440.0 | −78.35 | 204.0 | −442.5 | −73.08 |
| Min, Max | 35.0, 191.0 | −756, −138 | −94.1, −56.6 | 35.0, 626.0 | −1063, −188 | −96.3, −43.4 | 49.0, 668.0 | −752, 110.0 | −91.1, 19.7 |

TABLE 5(b)
Changes from baseline in Urine NTX:

| Cohort A | | | Cohort B | | | Cohort C | | |
|---|---|---|---|---|---|---|---|---|
| (N = 8) | | | (N = 11) | | | (N = 10) | | |
| | | % | | | % | | | % |
| Actual | Change | Change | Actual | Change | Change | Actual | Change | Change |

Baseline

| | | | |
|---|---|---|---|
| N | 8 | 11 | 10 |
| Mean | 175.6 | 81.5 | 127.1 |
| SD | 309.10 | 36.40 | 70.50 |
| Median | 65.5 | 70.0 | 127.0 |
| Min, Max | 29, 937 | 32, 133 | 28, 264 |

Day 7

| | | | | | | | | | |
|---|---|---|---|---|---|---|---|---|---|
| N | 8 | 8 | 8 | 11 | 11 | 11 | 10 | 10 | 10 |
| Mean | 40.4 | −135.3 | −76.96 | 26.1 | −55.4 | −67.42 | 23.3 | −103.8 | −78.45 |
| SD | 76.74 | 232.62 | 11.189 | 15.57 | 24.90 | 13.324 | 9.82 | 65.01 | 8.272 |
| Median | 13.5 | −53.5 | −80.20 | 24.0 | −64.0 | −64.66 | 22.5 | −98.0 | −81.19 |
| Min, Max | 7, 230 | −707, −15 | −86.6, −51.7 | 6, 53 | −86, −13 | −91.4, −40.6 | 10, 42 | −239, −18 | −90.5, −64.3 |

Day 56/Early Termination

| | | | | | | | | | |
|---|---|---|---|---|---|---|---|---|---|
| N | 7 | 7 | 7 | 11 | 11 | 11 | 10 | 10 | 10 |
| Mean | 21.4 | −45.4 | −66.74 | 22.0 | −59.5 | −67.48 | 43.6 | −83.5 | −62.01 |
| SD | 14.03 | 22.19 | 14.019 | 8.45 | 33.86 | 21.780 | 42.97 | 73.38 | 31.177 |
| Median | 17.0 | −43.0 | −74.14 | 21.0 | −63.0 | −73.68 | 27.5 | −94.5 | −65.84 |
| Min, Max | 10, 52 | −76, −12 | −81.7, −41.4 | 6, 36 | −107, −7 | −91.4, −18.4 | 14, 155 | −237, 26 | −89.8, 20.2 |

TABLE 5(c)
Changes from baseline in Serum Calcium:

| Cohort A | | | Cohort B | | | Cohort C | | |
|---|---|---|---|---|---|---|---|---|
| (N = 8) | | | (N = 11) | | | (N = 10) | | |
| | | % | | | % | | | % |
| Actual | Change | Change | Actual | Change | Change | Actual | Change | Change |

Baseline

| | | | |
|---|---|---|---|
| N | 8 | 11 | 10 |
| Mean | 9.4 | 9.4 | 9.6 |
| SD | 0.46 | 0.25 | 0.49 |
| Median | 9.4 | 9.4 | 9.7 |
| Min, Max | 8, 10 | 9, 10 | 9, 10 |

Day 7

| | | | | | | | | | |
|---|---|---|---|---|---|---|---|---|---|
| N | 8 | 8 | 8 | 11 | 11 | 11 | 10 | 10 | 10 |
| Mean | 8.8 | −0.5 | −5.40 | 8.8 | −0.6 | −6.75 | 8.8 | −0.8 | −7.89 |
| SD | 0.42 | 0.27 | 2.927 | 0.31 | 0.35 | 3.648 | 0.50 | 0.45 | 4.513 |
| Median | 8.9 | −0.5 | −5.57 | 8.7 | −0.8 | −8.02 | 8.7 | −0.8 | −8.40 |
| Min, Max | 8, 9 | −1, 0 | −9.6, 0 | 8, 10 | −1, 0 | −10.8, 2.6 | 8, 10 | −1, 0 | −12.9, 1.1 |

Day 56/Early Termination

| | | | | | | | | | |
|---|---|---|---|---|---|---|---|---|---|
| N | 6 | 6 | 6 | 11 | 11 | 11 | 10 | 10 | 10 |
| Mean | 9.0 | −0.5 | −5.16 | 8.6 | −0.8 | −8.24 | 8.9 | −0.6 | −6.46 |
| SD | 0.60 | 0.47 | 5.115 | 0.32 | 0.29 | 3.087 | 0.33 | 0.39 | 3.947 |
| Median | 9.0 | −0.4 | −4.65 | 8.6 | −0.8 | −8.25 | 9.0 | −0.7 | −7.03 |
| Min, Max | 8, 10 | −1, 0 | −13.2, 0.9 | 8, 9 | −1, 0 | −13.7, −2.1 | 8, 9 | −1, 0 | −12.9, 2.3 |

TABLE 5(d)
Changes from baseline in bone alkaline phosphatase (BAP):

| Cohort A | | | Cohort B | | | Cohort C | | |
|---|---|---|---|---|---|---|---|---|
| (N = 8) | | | (N = 11) | | | (N = 10) | | |
| | | % | | | % | | | % |
| Actual | Change | Change | Actual | Change | Change | Actual | Change | Change |

Baseline

| | | | |
|---|---|---|---|
| N | 8 | 11 | 10 |
| Mean | 55.8 | 32.0 | 46.0 |
| SD | 99.30 | 16.93 | 27.72 |
| Median | 17.9 | 37.7 | 40.0 |
| Min, Max | 7, 300 | 13, 63 | 11, 102 |

Day 7

| | | | | | | | | | |
|---|---|---|---|---|---|---|---|---|---|
| N | 8 | 8 | 8 | 11 | 11 | 11 | 10 | 10 | 10 |
| Mean | 44.1 | −11.6 | −9.58 | 30.5 | −1.6 | −4.82 | 40.8 | −5.2 | −10.37 |
| SD | 68.45 | 32.76 | 19.375 | 16.29 | 3.28 | 11.891 | 25.33 | 10.26 | 17.893 |
| Median | 15.5 | −1.3 | −8.25 | 34.3 | −3.4 | −8.93 | 35.6 | −5.3 | −17.43 |
| Min, Max | 7, 208 | −91, 14 | −30.5, 28.7 | 11, 57 | −6, 5 | −24.8, 12.8 | 12, 89 | −26, 10 | −29.5, 13.7 |

Day 56/Early Termination

| | | | | | | | | | |
|---|---|---|---|---|---|---|---|---|---|
| N | 7 | 7 | 7 | 11 | 11 | 11 | 10 | 10 | 10 |
| Mean | 19.8 | −1.1 | −18.08 | 35.0 | 2.9 | −4.46 | 52.6 | 6.6 | 15.60 |
| SD | 20.75 | 9.18 | 30.358 | 32.00 | 18.81 | 40.795 | 36.94 | 24.81 | 56.766 |
| Median | 14.4 | −2.8 | −32.85 | 25.2 | −3.6 | −27.07 | 48.6 | −1.2 | −5.14 |
| Min, Max | 4, 65 | −9, 18 | −43.5, 38.9 | 8, 113 | −17, 50 | −40.4, 79.9 | 10, 122 | −18, 47 | −55.2, 118.0 |

### (2) Secondary Efficacy: Brief Pain Inventory for Cohort A, B and C

The Brief Pain Inventory (BPI) Short Form data is illustrated in FIGS. 6(a) and 6(b). As shown in FIGS. 6(a) and 6(b), compared to Cohorts A and C, Cohort B showed superiority in the change from baseline responses in the worst and least pain, and pain scores.

### Example 3

### Studies on Adverse Effects (AE) of Patients Administered Bisphosphonates Under Cohort A, B and C

Studies of the impacts of the dosage schedule on adverse effects (AE) were conducted in the clinical trial described in Example 2. A study comparing two dosage regimens Orazol™ (cohort B and C) with standard IV Zometa® (cohort A) over 2 month was conducted. The study of the adverse effects for the three dosage regimens is discussed below.

### (1) Display of Adverse Effect

A total of 42 adverse events were reported by 18 of 30 patients who participated in the study. Of patients experiencing at least 1 event, 6 of 8 (75%) occurred in Cohort A, 5 of 11 (46%) occurred in Cohort B, and 7 of 11 (64%) in Cohort C.

A summary of adverse effects by system organ class of Cohort A, B and C are presented in Table 6. For all patients, 18 of 30 (60%) experienced ≧1 AE during the study. Nine of 30 (30%) patients experienced ≧1 AE related to musculoskeletal and connective tissue disorders, with bone pain as the most commonly reported event (7 of 9, 73%). Eight of 30 (27%) patients experienced ≧1 AE in the general disorders and administration site conditions class, with pyrexia the most commonly reported event (5 of 8 patients, 17%).

The most commonly reported adverse events were classified as musculoskeletal and connective tissue disorders, reported by 9 of 30 (30%) patients: 3 (38%) in Cohort A, 2 (18%) in Cohort B, and 4 (36%) in Cohort C. Bone pain was reported by patients in each cohort: 3 patients (38%) in Cohort A, 2 (18%) in Cohort B, and 2 (18%) in Cohort C. Therefore, the patients under Cohort B has the lowest percentage of reported AE for musculoskeletal, connective tissue disorders, and bone pain.

TABLE 6
Summary of Adverse Events by System Organ Class (Safety Population)

| | System Organ Class | | | |
|---|---|---|---|---|
| | Cohort A | Cohort B | Cohort C | All Patients |
| | N = 8 | N = 11 | N = 11 | N = 30 |
| Preferred Term | n (%) | n (%) | n (%) | n (%) |
| Number of Patients with ≧1 AE | 6 (75.0) | 5 (45.5) | 7 (63.6) | 18 (60.0) |
| Gastrointestinal disorders | 0 | 1 (9.1) | 1 (9.1) | 2 (6.7) |
| Abdominal pain upper | 0 | 1 (9.1) | 0 | 1 (3.3) |
| Diarrhea | 0 | 0 | 1 (9.1) | 1 (3.3) |
| Nausea | 0 | 0 | 1 (9.1) | 1 (3.3) |
| General disorders and administration site | 4 (50.0) | 2 (18.2) | 2 (18.2) | 8 (26.7)) |
| conditions | | | | |
| Fatigue | 0 | 2 (18.2) | 0 | 2 (6.7) |
| Edema peripheral | 0 | 0 | 1 (9.1) | 1 (3.3) |
| Pyrexia | 4 (50.0) | 0 | 1 (9.1) | 5 (16.7) |
| Infections and infestations | 0 | 0 | 2 (18.2) | 2 (6.7) |
| Herpes zoster | 0 | 0 | 1 (9.1) | 1 (3.3) |
| Influenza | 0 | 0 | 1 (9.1) | 1 (3.3) |
| Musculoskeletal and connective tissue disorders | 3 (37.5) | 2 (18.2) | 4 (36.4) | 9 (30.0) |
| Arthralgia | 0 | 0 | 1 (9.1) | 1 (3.3) |
| Bone pain | 3 (37.5) | 2 (18.2) | 2 (18.2) | 7 (73.3) |
| Musculoskeletal chest pain | 0 | 0 | 1 (9.1) | 1 (3.3) |
| Musculoskeletal pain | 0 | 0 | 1 (9.1) | 1 (3.3) |
| Myalgia | 1 (12.5) | 0 | 1 (9.1) | 2 (6.7) |
| Nervous system disorders | 1 (12.5) | 0 | 0 | 1 (3.3) |
| Headache | 1 (12.5) | 0 | 0 | 1 (3.3) |
| Renal and urinary disorders | 0 | 0 | 1 (9.1) | 1 (3.3) |
| Urinary retention | 0 | 0 | 1 (9.1) | 1 (3.3) |
| Respiratory, thoracic and mediastinal disorders | 0 | 2 (18.2) | 0 | 2 (6.7) |
| Dyspnea | 0 | 1 (0.1) | 0 | 1 (3.3) |
| Nasopharyngitis | 0 | 1 (9.1) | 0 | 1 (3.3) |

Cohort A = IV Zometa 4 mg, 15-minute infusion, Day 0 and Day 28;
Cohort B = Orazol, 20 mg, Days 0, 7, 14, 21, 28, 35, 42, and 49;
Cohort C = Orazol, 20 mg, Days 0, 1, 2, 3, 28, 35, 42, and 49.

### (2) Display of Adverse Events by Body System, Preferred Dosage Schedule, and Maximum Severity

The incidence of all AEs by severity that occurred during the treatment period in the safety population is presented in Table 7

TABLE 7
Adverse Events by System Organ Class, Preferred Term, and Maximum Severity
Safety Population

| System Organ | | Cohort A | Cohort B | Cohort C | All Patients |
|---|---|---|---|---|---|
| Class Preferred term | Severity | (N = 8) | (N = 11) | (N = 11) | (N = 30) |
| Number of | Mild | 2 (25.0) | 3 (27.3) | 1 (9.1) | 6 (20.0) |
| Patients with >=1 AE | Moderate | 3 (37.5) | 2 (18.2) | 5 (45.5) | 10 (33.3) |
| | Severe | 1 (12.5) | 0 | 1 (9.1) | 2 (6.7) |
| Gastrointestinal disorders | Mild | 0 | 0 | 0 | 0 |
| | Moderate | 0 | 1 (9.1) | 1 (9.1) | 2 (6.7) |
| | Severe | 0 | 0 | 0 | 0 |
| Abdominal pain upper | Mild | 0 | 0 | 0 | 0 |
| | Moderate | 0 | 1 (9.1) | 0 | 1 (3.3) |
| | Severe | 0 | 0 | 0 | 0 |
| Diarrhoea | Mild | 0 | 0 | 0 | 0 |
| | Moderate | 0 | 0 | 1 (9.1) | 1 (3.3) |
| | Severe | 0 | 0 | 0 | 0 |
| Nausea | Mild | 0 | 0 | 0 | 0 |
| | Moderate | 0 | 0 | 1(9.1) | 1 (3.3) |
| | Severe | 0 | 0 | 0 | 0 |
| General disorders and | Mild | 2 (25.0) | 1 (9.1) | 1 (9.1) | 4 (13.3) |
| administration site | Moderate | 2 (25.0) | 1 (9.1) | 1 (9.1) | 4 (13.3) |
| conditions: | Severe | 0 | 0 | 0 | 0 |
| Fatigue | Mild | 0 | 1 (9.1) | 0 | 1 (3.3) |
| | Moderate | 0 | 1 (9.1) | 0 | 1 (3.3) |
| | Severe | 0 | 0 | 0 | 0 |
| Oedema | Mild | 0 | 0 | 0 | 0 |
| peripheral | Moderate | 0 | 0 | 1 (9.1) | 1 (3.3) |
| | Severe | 0 | 0 | 0 | 0 |
| Pyrexia | Mild | 2 (25.0) | 0 | 1 (9.1) | 3 (10.0) |
| | Moderate | 2 (25.0) | 0 | 0 | 2 (6.7) |
| | Severe | 0 | 0 | 0 | 0 |
| Infections and | Mild | 0 | 0 | 2 (18.2) | 2 (6.7) |
| infestations | Moderate | 0 | 0 | 0 | 0 |
| | Severe | 0 | 0 | 0 | 0 |
| Herpes zoster | Mild | 0 | 0 | 1 (9.1) | 1 (3.3) |
| | Moderate | 0 | 0 | 0 | 0 |
| | Severe | 0 | 0 | 0 | 0 |
| Influenza | Mild | 0 | 0 | 1 (9.1) | 1 (3.3) |
| | Moderate | 0 | 0 | 0 | 0 |
| | Severe | 0 | 0 | 0 | 0 |
| Musculoskeletal | Mild | 1 (12.5) | 2 (18.2) | 0 | 3 (10.0) |
| and connective | Moderate | 1 (12.5) | 0 | 3 (27.3) | 4 (13.3) |
| tissue disorders | Severe | 1 (12.5) | 0 | 1 (9.1) | 2 (6.7) |
| Arthralgia | Mild | 0 | 0 | 0 | 0 |
| | Moderate | 0 | 0 | 1 (9.1) | 1 (3.3) |
| | Severe | 0 | 0 | 0 | 0 |
| Bone pain | Mild | 1 (12.5) | 2 (18.2) | 0 | 3 (10.0) |
| | Moderate | 1 (12.5) | 0 | 2 (18.2) | 3 (10.0) |
| | Severe | 1 (12.5) | 0 | 0 | 1 (3.3) |
| Musculoskeletal | Mild | 0 | 0 | 1 (9.1) | 1 (3.3) |
| chest pain | Moderate | 0 | 0 | 0 | 0 |
| | Severe | 0 | 0 | 0 | 0 |
| Musculoskeletal | Mild | 0 | 0 | 0 | 0 |
| pain | Moderate | 0 | 0 | 0 | 0 |
| | Severe | 0 | 0 | 1 (9.1) | 1 (3.3) |
| Myalgia | Mild | 1 (12.5) | 0 | 0 | 1 (3.3) |
| | Moderate | 0 | 0 | 1 (9.1) | 1 (3.3) |
| | Severe | 0 | 0 | 0 | 0 |
| Nervous system | Mild | 0 | 0 | 0 | 0 |
| disorders | Moderate | 1 (12.5) | 0 | 0 | 1 (3.3) |
| | Severe | 0 | 0 | 0 | 0 |
| Headache | Mild | 0 | 0 | 0 | 0 |
| | Moderate | 1 (12.5) | 0 | 0 | 1 (3.3) |
| | Severe | 0 | 0 | 0 | 0 |
| Renal and urinary | Mild | 0 | 0 | 0 | 0 |
| disorders | Moderate | 0 | 0 | 1 (9.1) | 1 (3.3) |
| | Severe | 0 | 0 | 0 | 0 |
| Urinary retention | Mild | 0 | 0 | 0 | 0 |
| | Moderate | 0 | 0 | 1 (9.1) | 1 (3.3) |
| | Severe | 0 | 0 | 0 | 0 |
| Respiratory, | Mild | 0 | 1 (9.1) | 0 | 1 (3.3) |
| thoracic and | Moderate | 0 | 1 (9.1) | 0 | 1 (3.3) |
| mediastinal | Severe | 0 | 0 | 0 | 0 |
| disorders | | | | | |
| Dyspnoea | Mild | 0 | 0 | 0 | 0 |
| | Moderate | 0 | 1 (9.1) | 0 | 1 (3.3) |
| | Severe | 0 | 0 | 0 | 0 |
| Nasopharyngitis | Mild | 0 | 1 (9.1) | 0 | 1 (3.3) |
| | Moderate | 0 | 0 | 0 | 0 |
| | Severe | 0 | 0 | 0 | 0 |

Cohort A = IV Zometa 4 mg, 15-minute infusion, Day 0 and Day 28;
Cohort B = MER-101 po, 20 mg, Days 0, 7, 14, 21, 28, 35, 42, and 49;
Cohort C = MER-101 po, 20 mg, Days 0, 1, 2, 3, 28, 35, 42, and 49.

As shown in Table 7, of the 18 patients who experienced ≧1 AE, 6 patients (20%) reported maximum severity of events described as mild, 10 patients (33%) reported maximum severity of events described as moderate, and 2 patients (6.7%) reported events described as severe.

Regarding maximum severity per cohort:

In Cohort A, 2 (25%) patients experienced ≧1 AE that was mild, 3 (38%) experienced ≧1 AE that was moderate, and 1 (13%) experienced ≧1 AE that was severe.

In Cohort B, 3 (27%) patients experienced ≧1 AE that was mild and 2 (18%) patients experienced ≧1 AE that was moderate. No events were severe.

In Cohort C, 1 (9%) patient experienced ≧1 AE that was mild, 5 (46%) patients experience ≧1 event that was moderate, and 1 (9%) experienced ≧1 AE that was severe in intensity.

Compared to Cohorts A and C, patients under Cohort B have reported the least severity of the adverse effect.

### (3) Adverse Events by Relationship to Study Drug

A summary of AEs and their relationship to study drug is provided in Table 8. For all patients, 10 (33%) experienced ≧1 AE that was deemed not related to study drug and 8 (27%) patients experienced ≧1 AE that was suspected to be related. As shown in Table 8, the greatest proportion of patients with AEs suspected to be related to study drug was found in Cohort A (50%). Compared to cohort A or C, Cohort B has the least number of AEs that are suspected to be related to drug.

TABLE 8
Study Number of Patients Experiencing ≧1 Adverse Event
by Relationship to Study Medication (Safety Population)

| | | Number of Patients | Not Related | Related |
|---|---|---|---|---|
| Cohort | (N) | n (%) | n (%) | n (%) |
| A | (8) | 6 (75%) | 2 (25%) | 4 (50%) |
| B | (11) | 5 (45%) | 4 (36%) | 1 (9%) |
| C | (11) | 7 (64%) | 4 (36%) | 3 (27%) |

A summary of AEs that were suspected to be related to study drug are summarized by cohort and preferred term in Table 9. As shown in Table 9, the patients under Cohort B have no reported acute phase reactions such as fever, muscle pain, or bone pain.

TABLE 9
Summary of Adverse Events Suspected to be Related to Study
Medication by Cohort and Preferred Term (Safety Population)

| | Adverse Events | | |
|---|---|---|---|
| | | Number | |
| | | of | |
| Cohort | Event | Events | Comments |
| A | fever | 7 | reported by 4 patients - all onsets |
| | | | within 24 hours after dosing |
| | headache | 2 | reported by 1 patient - both onsets |
| | | | within 24 hours after dosing |
| | bone pain | 1 | reported by 1 patient - onset within 24 |
| | | | hours after dosing |
| | muscle pain | 1 | reported by 1 patient - onset within 24 |
| | | | hours after dosing |
| B | stomach pain | 5 | reported by 1 patient.- 4 onsets on the |
| | | | day after dosing |
| | fatigue | 1 | reported by 1 patient - began after 5^{th} |
| | | | dose and was ongoing |
| C | nausea | 3 | reported by 1 patient - duration of 4- |
| | | | day loading dose and doses 5 and 6 |
| | diarrhea | 2 | reported by 1 patient - onsets within |
| | | | 24 hours after doses 5 and 6 |
| | fever | 1 | reported by 1 patient - on days 2-4 of |
| | | | loading dose |
| | bone pain | 1 | reported by 1 patient - on days 2-4 of |
| | | | loading dose |
| | muscle pain | 1 | reported by 1 patient - on days 2-4 of |
| | | | loading dose |
| | pain in ribs | 1 | reported by 1 patient - onset on Day 2 |
| | and sternum | | of the 4 day loading dose/hospitalized |

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method of treating or preventing a medical condition that is responsive to a bisphosphonate compound in a subject, the method comprising:
administering to the subject a pharmaceutical composition comprising a therapeutically effective amount of the bisphosphonate no less frequently than a bi-weekly dosage schedule, wherein the bisphosphonate compound is zoledronic acid.

2. The method of claim 1, wherein the bisphosphonate is administered to the subject via intravenous administration.

3. The method of claim 1, wherein the bisphosphonate is orally administered to the subject.

4. The method of claim 1, wherein the treatment or prevention provides sustained therapeutic effects of the bisphosphonate.

5. The method of claim 4, wherein the level of N-Telopeptide Cross-Links (NTX) in urine of the subject is decreased and maintained in a range of about 5 to about 60 BCE/mMol during the treatment.

6. The method of claim 4, wherein the level of serum C-telopeptide (CTX) of the subject is decreased and maintained at a range of about 35 to about 600 pg/mL during the treatment.

7. The method of claim 1, wherein the treatment or prevention provides reduced adverse effects resulting from administering a bisphosphonate compound to the subject comparing to the treatment of administering bisphosphonate compound via IV infusion or orally administration on a monthly or yearly dosage schedule.

8. The method of claim 7, wherein the adverse effects are selected from the group consisting of renal damage, general malaise, acute phase reaction, stomach pain, fatigue, nausea, and a combination thereof.

9. The method of claim 8, wherein the acute phase reaction is selected from the group consisting of fever, muscle pain, bone pain and a combination thereof.

10. The method of claim 1, wherein the bisphosphonate is administered to the subject on a weekly dosage schedule.

11. The method of claim 1, wherein the bisphosphonate is administered to the subject on a daily dosage schedule.

12. The method of claim 1, wherein the pharmaceutical composition is administered orally, and the oral dose of the bisphosphonate compound is about 8 to 400 times more than the systemic dose of bisphosphonate compound administered through intravenous infusion.

13. The method of claim 1, wherein the medical condition is selected from the group consisting of osteoporosis, rheumatoid arthritis, bone fracture, excessive bone resorption and a combination thereof.

14. The method of claim 13, wherein the systemic dose of the pharmaceutical composition is in a range of about 0.000018 mmol to about 0.00015 mmol of the bisphosphonate compound per day.

15. The method of claim 13, wherein the systemic dose of the pharmaceutical composition is in a range of about 0.00013 mmol to about 0.001 mmol of the bisphosphonate compound per week.

16. The method of claim 1, wherein the medical condition is selected from the group consisting of systemic lupus erythematosus (SLE), cancer, tumor induced hypocalcemia, bone metastasis and a combination thereof.

17. The method of claim 16, wherein the cancer is selected from the group consisting of prostate cancer, metastatic bone cancer, lung cancer, multiple myeloma breast cancer and any solid tumor that induces metastatic disease.

18. The method of claim 16, wherein the systemic dose of the pharmaceutical composition is in a range of about 0.00018 mmol to about 0.0015 mmol of the bisphosphonate compound per day.

19. The method of claim 16, wherein the systemic dose of the pharmaceutical composition is in a range of about 0.0013 mmol to about 0.01 mmol of the bisphosphonate compound per week.

20. The method of claim 1, wherein the pharmaceutical composition is in a solid oral dosage form.

21. The method of claim 1, wherein the pharmaceutical composition further comprises an enhancer, wherein said enhancer is a medium chain fatty acid salt, an ester, an ether, or a derivative of a medium chain fatty acid and has a carbon chain length of from about 4 to about 20 carbon atoms.

22. The method of claim 21, wherein the carbon chain length of the enhancer is from 6 to 20 carbon atoms.

23. The method of claim 21, wherein the carbon chain length is from 8 to 14 carbon atoms.

24. The method of claim 21, wherein the enhancer is a sodium salt of a medium chain fatty acid.

25. The method of claim 21, wherein the enhancer is selected from the group consisting of sodium caprylate, sodium caprate, sodium laurate and a combination thereof.

26. The method of claims 21, wherein the enhancer is sodium caprate.

27. The method of claim 21, wherein the bisphosphonate and the enhancer are present in a ratio of from 1:100,000 to 10:1 (bisphosphonate:enhancer).

28. The method of claim 21, wherein the composition is in the form of a delayed release enteric coated tablet.
